(19) United States
(12) Patent Application Publication
(10) Pub. No.: US 2025/0319143 A1
(43) Pub. Date: Oct. 16, 2025

(54) BACTERIOPHAGE PREPARATION IN THE FORM OF GEL TO PREVENT OR TREAT BACTERIAL INFECTIONS IN DAIRY CATTLE, ITS MANUFACTURING METHOD AND BACTERIOPHAGE STRAINS

(71) Applicant: Proteon Pharmaceuticals S.A., Lódz (PL)

(72) Inventors: Jaroslaw DASTYCH, Lódz (PL); Ewelina A. WÓJCIK, Grabica (PL); Agnieszka MASZEWSKA, Lódz (PL); Marta SZYMANSKA, Lódz (PL); Przemyslaw SZEWCZYK, Lódz (PL); Elzbieta FORNAL, Konstantynów Lódzki (PL); Joanna KAZIMIERCZAK, Lódz (PL); Arkadiusz GUZINSKI, Lódz (PL); Marta KRZYZANIAK, Lódz (PL); Daria KRÓLIKOWSKA, Lódz (PL); Justyna ANDRYSIAK, Lódz (PL); Paulina WIGNER-JEZIORSKA, Lódz (PL); Malgorzata STANCZYK, Lódz (PL)

(73) Assignee: Proteon Pharmaceuticals S.A., Lódz (PL)

(21) Appl. No.: 18/286,377

(22) PCT Filed: Apr. 11, 2022
(86) PCT No.: PCT/PL2022/050021

§ 371 Date: Oct. 11, 2023

(30) Foreign Application Priority Data
Apr. 11, 2021 (PL) P.437549

(51) Int. Cl. A61K 35/76; A61K 9/00; A61K 9/06; A61K 47/36; A61P 31/04

(52) U.S. Cl. CPC A61K 35/76; A61K 9/0041; A61K 9/06; A61K 47/36; A61P 31/04

(57) ABSTRACT

A bacteriophage preparation in the form of gel to prevent or treat bacterial infections in dairy cattle, including but not limited to infections caused by E. coli and/or S. aureus, intended for intramammary administration during lactation or dry period, in particular, to treat or prevent mastitis in dairy cattle, its manufacturing method and bacteriophage strains especially useful for such preparation manufacturing, characterised in detail in the enclosed patent claims, was revealed.

The presented invention applies to a bacteriophage preparation in the form of gel to prevent or treat bacterial infections in dairy cattle, including but not limited to infections caused by E. coli and/or S. aureus, intended for intramammary administration during lactation or dry period, in particular, to treat or prevent mastitis in dairy cattle, its manufacturing method and bacteriophage strains especially useful for such a preparation manufacturing.

Cow's milk optimum production, which amounts to nearly 81% of the entire milk production, is mainly restrained by mastitis. Economic losses resulting from the costs of mastitis treatment include the decrease in the quality and quantity of the produced milk and an increase in the slaughter indicator or mortality of productive animals. The main etiological factors of mastitis in cows include Staphylococcus aureus and Escherichia coli bacteria [Schukken Y. H. et al., 2011]. Mastitis treatment, which requires maintaining milk cure period, typically includes treatment with intramammary or—rarely—intramuscular antibiotics during dry period or lactation [Kuipers A. et al., 2016; Burmańczuk A. et al., 2017; Crispie F. et al., 2004]. Unfortunately, the commonly used antibiotic therapy contributes to the occurrence of antibiotic-resistant bacteria strains [Gomes F. et al., 2016; Teale and David, 1999]. Recurrent infections, making up for 40% of all mastitis cases in cattle, possibly resulting from the bacteria forming a biofilm, are another important problem [Hillerton J. E. and Kliem K. E., 2002; Vasudevan P. et al., 2003]. The bacteria in the biofilm are 10-1,000 times more resistant to antibiotics, and the biofilm environment fosters genetic modifications in the bacteria, leading to their losing sensitivity to antibiotics [Melchior M. B. et al., 2006; Łubowska N. and Piechowicz L., 2018]. E. coli and S. aureus strains that cause mastitis are capable of forming a biofilm [Raza A. et al., 2013, Costa J. C. M. et al., 2014].

A continuous rise in morbidity cases, legislative constraints in using antibiotics in animal breeding imposed by the EU, and high drug-resistance of S. aureus and E. coli strains force the development of alternative methods to control the pathogens in cattle breeding. The use of bacteriophages (phages)—viruses capable of destroying specific bacteria groups—can be one of the methods. A phage therapy (phagotheraphy), contrary to antibiotic therapy, is characterised by high specificity and no side effects [Matsuzaki S. et al., 2003]. Moreover, phages multiply exponentially, and that is why an application of a single dose may treat the infection [Yu Y. P., 2013; Elbreki M. et al., 2014; Golkar Z. et al., 2014]. Using an adequately composed phage cocktail ensures activity against a broader spectrum of bacterial strains and helps avoid problems of bacteria resistance to the applied phages, minimising the risk of phage therapy inefficacy [Li Z. et al., 2016; Brüssow H., 2005]. The highest efficacy of multiphage preparations in mastitis development prevention was observed when they contained at least 3 or 4 bacteriophages [Patent US20030235560A1].

Previous studies revealed that vB_SauS IMEP5 [Zhang Q. et al., 2017], ΦH5 and ΦA72 [García P. et al., 2009], SAP-1 and SAP-3 [Soo Son J. et al., 2010], vB_SauM_JS25 [Zhang L. et al., 2015], SPW [Li L. and Zhang Z., 2014], SAH-1 [Han J. E. et al., 2013], Ufv-aur2, Ufv-aur5, Ufv-aur6, Ufv-aur11, Ufv-aur3 and Ufv-aur7 [Dias R. S. et al., 2013] and OMSP bacteriophages [Sangha K. K. et al., 2014] demonstrate lytic activity towards S. aureus strains isolated from cows with mastitis. Moreover, SAH-1 bacteriophage reduced the growth of S. aureus (including methicillin-resistant ones), isolated from cows with mastitis in a dry period [Han J. E. et al., 2013]. Similarly, applying a mixture of three phages caused lysis of S. aureus strains, including those resistant to the tested antibiotics [Varela-Ortiz D. F. et al., 2018]. The growth of E. coli strains isolated from diseased cattle was inhibited by a cocktail containing four phages similar to T4, related to rV5 phage and phi92 [Porter J. et al., 2016]. Horiuk Y. V. et al. [2019] demonstrated that SAvB14 phage caused a 30-times reduction in the number of S. aureus cells in a young biofilm. Mastitis therapy can also be based on the application of isolated bacteriophage lytic enzymes [Patent No. CA2661896]. Bacteriophage polysaccharide depolymerases can be used for treating infections related to biofilm formation [Horiuk Y. V. et al., 2019]. The available literature also reveals a synergistic action of phages and antibiotics inhibiting the S. aureus in vitro growth [Kumaran D. et al., 2018; Tkhilaishvili T. et al., 2018; Rahman M. et al., 2011]. Bacteriophages used individually (vB_EcoM-UFV017 phage), in a mixture (BECP2 and BECP6 phages) or with antibiotics (T4 phage and cefotaxime) hampered biofilm formation by E. coli, including the strains isolated from cows with mastitis [Ribeiro K. V. G. et al., 2018; Lee Y. D., 2015; Ryan E. M. et al., 2012].

In vivo tests based on murine mastitis model confirmed the efficacy of phages in treating mastitis caused by S. aureus and E. coli [Breyne K. et al., 2017; Geng H. et al., 2020; de Silva Durate V. et al., 2018]. ΦSA012 and ΦSA039 bacteriophages demonstrated a strong in vitro lytic activity towards several dozen S. aureus strains isolated from cattle with mastitis. Additionally, using a murine mastitis model, it was demonstrated that ΦSA012 phage reduced the bacteria count in the tissue and contributed to inflammation alleviation in the mammary glands [Iwano H. et al., 2018].

DW2, CS1 and K bacteriophages, specific to S. aureus, used as a cocktail for soaking or direct intramammary administration, prevented mastitis caused by S. aureus in cows [O'Flaherty S. et al., 2005]. The inhibition of S. aureus strains causing mastitis was observed both in vitro and in vivo (local spraying) conditions after applying SAP-2 phage [U.S. Pat. No. 8,043,613B2]. The intramammary administration during lactation and dry period of at least four phages similar to p0031, p0032, p0033, p0034 and p0045 were effective in treating mastitis caused by E. coli. The application of the cocktail in a gel form, combined with an intramammary-administered bismuth-based carrier, inhibited the growth of E. coli [Patent No. WO2017087909A1]. The therapeutic potential in mastitis treatment was also revealed by compositions containing SA7 or SA3 bacteriophage specific to S. aureus, administered orally as a feed additive and a disinfecting or cleaning agent [Patent No. KR20180042748A; Patent No. KR102003770B1].

Despite the high antibacterial potential of the phages, the use of bacteriophage preparations has some constraints. The bacteriophage application is among the constraints. In intravenous administration, only a small amount of the phages reach the target place due to the reticular-endothelial system's immunological activity. Similarly, because of the reduced consumption of dry matter by cows suffering from mastitis, the application of phage preparation in the form of feed additives can be ineffective [Mainau E. et al., 2014]. With regard to the above, and since the pathogens causing mastitis mainly penetrate through the teats, intramammary administration seems to be the effective and easy way of administration. The choice of the right delivery carrier for the phages is pivotal as well.

A preparation for intramammary administration should fulfil the following requirements:
the preparation should have a form of gel at a refrigeration temperature and partly liquefy at ca. 36° C.,
the preparation should be homogeneous and free of syneresis, i.e. water leakage from the gel during storage.

The preparation should maintain the above mentioned physical and chemical parameters for at least 12 months when stored at 2-8° C.

Moreover, the delivery carrier should not negatively affect the bacteriophages' activity. For technological reasons, the carrier's aqueous solution must be fully miscible with the bacteriophage cocktail at 36° C.

It is desired that the preparation's activity was at least 95% stable after 12 months of storage at 2-8° C.

It is also desired that the preparation maintained its activity in milk.

Simultaneously, a bacteriophage preparation for mastitis treatment should reveal adequate bactericidal activity towards the bacterial strains typically responsible for mastitis, including but not limited to Staphylococcus aureus and Escherichia coli strains. It should demonstrate high lytic activity and specificity to these strains. Moreover, a phage preparation should be characterised by high efficacy in preventing and destroying biofilm formed by the referenced bacteria, and its efficacy in the collagen matrix model reflecting the in vivo conditions should exceed 30%.

Unexpectedly, the application of this invention offers a solution to the presented problems.

### Essence of the Invention

A bacteriophage preparation in the form of gel to prevent or treat bacterial infections in dairy cattle, including but not limited to infections caused by E. coli and/or S. aureus, intended for intramammary administration during lactation or dry period, in particular, to treat or prevent mastitis in dairy cattle, its manufacturing method and bacteriophage strains especially useful for such preparation manufacturing, characterised in detail in the enclosed patent claims, is the subject of the invention.

### DETAILED DESCRIPTION OF THE INVENTION

A formulation of a bacteriophage preparation intended to prevent or treat bacterial infections in dairy cattle, including but not limited to infections caused by E. coli and/or S. aureus is the subject of the invention, whereby the manufactured preparation is intended for intramammary administration to the animals at risk of infection, in a form immobilised in iota-carrageenan, during lactation and dry period.

The disclosed method of formulating a bacteriophage preparation specific to a broad range of bacterial strains pathogenic for dairy cattle, preferably E. coli and/or S. aureus, characterised in that:
the preparation is developed based on an aqueous solution of iota-carrageenan at 0.4-8.0% w/v concentration and bacteriophage cocktail specific to E. coli and/or S. aureus bacteria strains, with at least 10⁷ PFU/mL count, preferably at least 10⁸ PFU/mL count, particularly preferably at least 4×10⁸ PFU/mL count, preferably mixed at 1:1 quantitative ratio,
the ingredients are mixed in sterile conditions, at 33° C. to 40° C., preferably at 36° C.

The revealed method is used in selecting the composition of a bacteriophage preparation, with a broad specificity spectrum to the mastitis-causing bacteria, preferably to E. coli and/or S. aureus pathogenic for dairy cattle, which is significant for industrial applications.

The application of a bacteriophage preparation containing at least three bacteriophages selected from the group including the following phages: 303Ecol101PP, 308Ecol101PP, 310Ecol104PP, 348Ecol098PP, 241Ecol014PP, 351Saur083PP, 355Saur083PP, 357Saur119PP to prevent and treat mastitis caused by bacteria, preferably E. coli and/or S. aureus pathogenic strains, in dairy cattle is another subject of the invention, whereby the manufactured preparation is intended for intramammary administration as a gel containing bacteriophages and iota-carrageenan.

Preferably, the bacteriophage preparation manufactured according to the invention reveals strong therapeutic action in mastitis treatment, preferably caused by E. coli and/or S. aureus strains, because it improves milk quality and mitigates clinical symptoms of mastitis.

Preferably, the bacteriophage preparation manufactured according to the invention reveals strong prophylactic action in mastitis prevention, as it protects against bacterial infections, preferably caused by E. coli and/or S. aureus strains.

Preferably, the controlled infection is an infection with pathogenic bacteria that cause mastitis in dairy cattle, including but not limited to E. coli and/or S. aureus strains, while bacteriophage strains revealed in this application and deposited according to the Budapest Treaty on 22 Jan. 2020 in the Polish Collection of Microorganisms (PCM) (address: Institute of Immunology and Experimental Therapy, Polish Academy of Sciences, ul. Weigla 12, 53-114 Wroclaw) at the following deposit numbers F/00152 (303Ecol101PP strain), F/00153 (308Ecol101PP strain), F/00154 (310Ecol104PP strain), F/00155 (348Ecol098PP strain), F/00151 (241Ecol014PP strain), F/00148 (351Saur083PP strain), F/00149 (355Saur083PP strain), and F/00150 (357Saur119PP strain) are used for making the preparation.

The following bacteriophage strains 303Ecol101PP, 308Ecol101PP, 310Ecol104PP, 348Ecol098PP, 241Ecol014PP, 351Saur083PP, 355Saur083PP, 357Saur119PP that control dairy cattle infections caused by pathogenic E. coli and/or S. aureus strains are another subject of the invention.

A bacteriophage preparation according to the invention is based on the ecosystem's natural ingredients and does not have a negative impact on organisms other than specific pathogenic bacteria.

The bacteriophage preparation guarantees that E. coli and/or S. aureus strains pathogenic only for dairy cattle are selectively limited.

The bacteriophage preparation is suitable for use in animal production, especially to control E. coli and/or S. aureus bacterial pathogens in dairy cattle farms.

Unexpectedly, the bacteriophage strains revealed in this claim have broad specificity involving lysis of 16 out of 18 different E. coli strains and all 15 tested S. aureus strains isolated from cows with mastitis.

The use of iota-carrageenan fraction as a gel carrier for intramammary administration of the phages is another subject of the invention. The bacteriophages disclosed in the claim, prepared as an iota-carrageenan gel, remain stable for at least 12 months at 4° C.

For a better explanation, the invention is illustrated in the enclosed Figures, where:

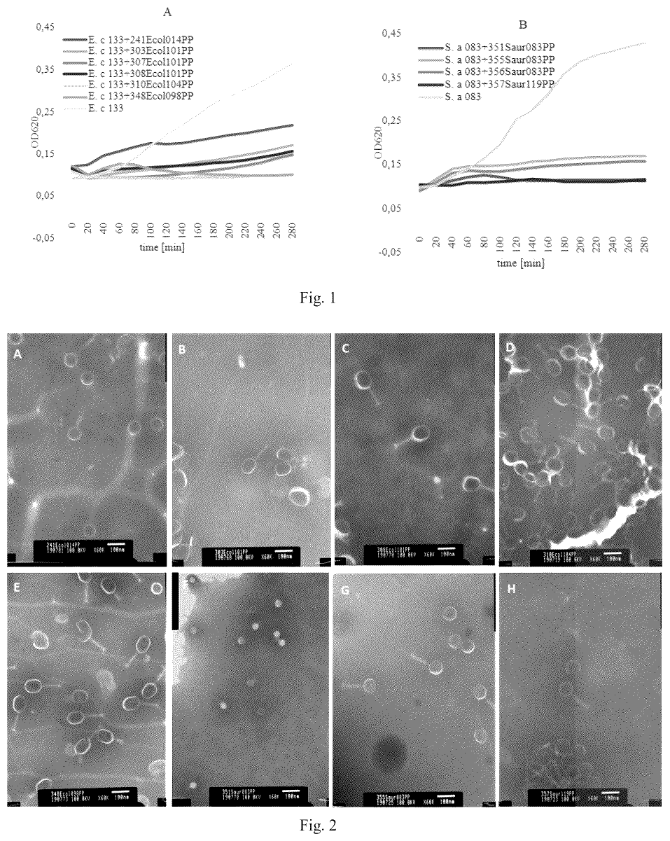

FIG. 1 shows the growth curves of E. coli_133 and S. aureus_083 treated with individual bacteriophages specific to the particular bacteria species.

FIG. 2 shows the morphology of the bacteriophages included in the bacteriophage preparation: A—241Ecol014PP; B—303Ecol101PP, C—308Ecol101PP, D—310Ecol104PP, E—348Ecol098PP, F—351Saur083PP, G—355Saur083PP, and H—357Saur119PP. 60,000× magnification.

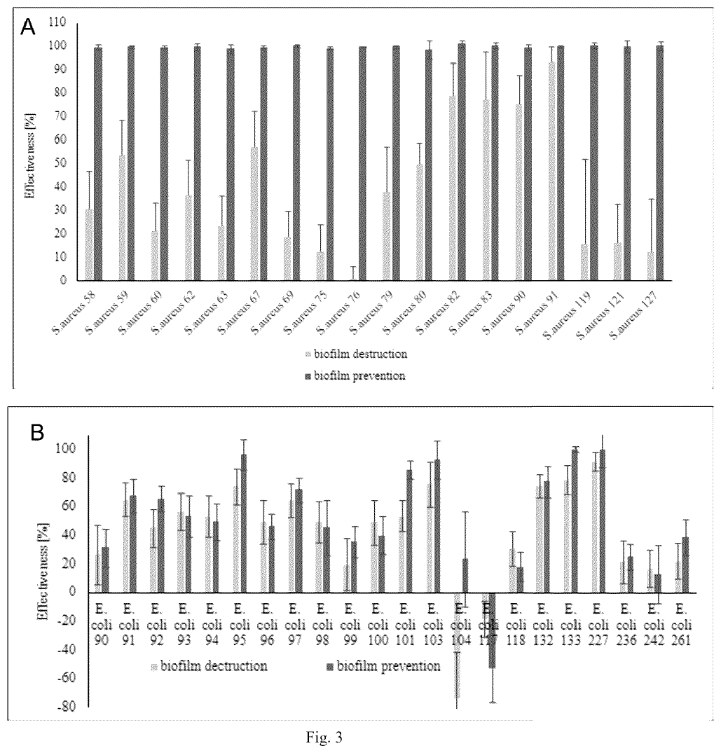

FIG. 3 shows the bacteriophage preparation influence on biofilm destruction and formation prevention.

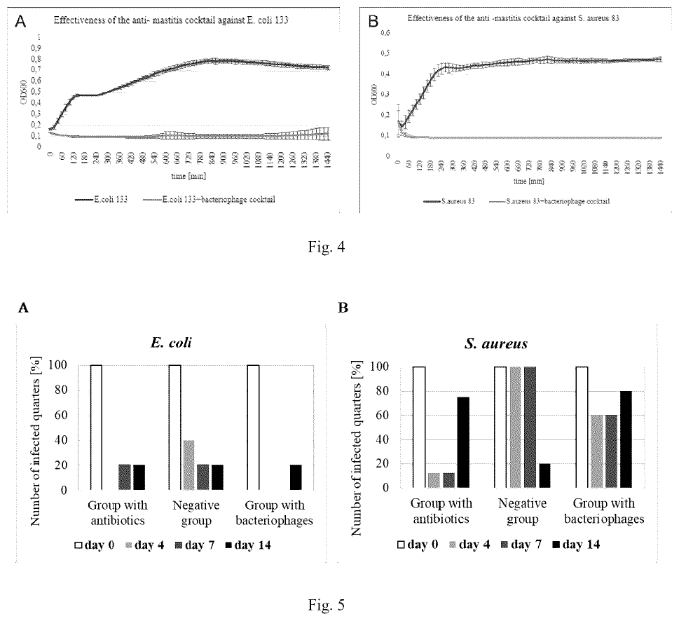

FIG. 4 shows the growth curves of E. coli_133 and S. aureus_083 treated with the developed bacteriophage cocktail.

FIG. 5 shows the influence of antibiotics and bacteriophage preparation on the number of udder quarters infected with E. coli and S. aureus.

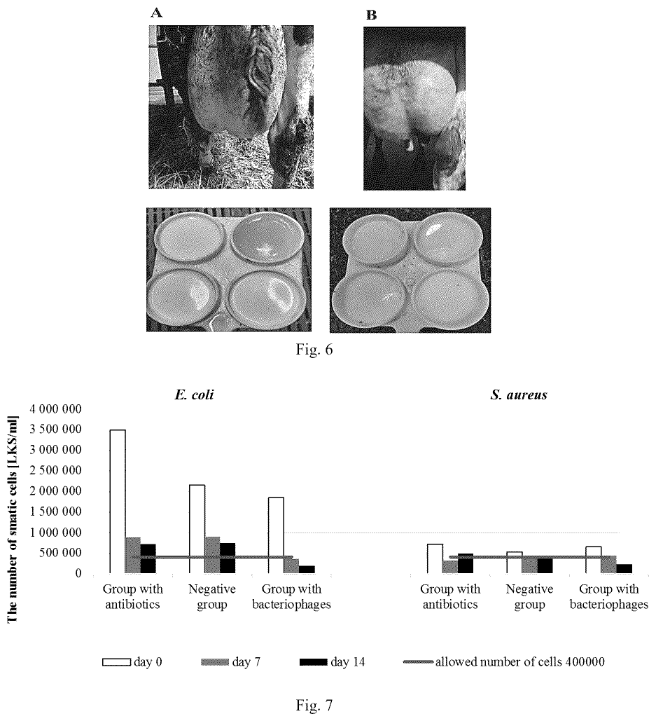

FIG. 6 shows clinical changes in the udders of dairy cows with mastitis caused by E. coli (left quarter not infected, right quarter infected) and milk before bacteriophage preparation administration on day 0 of the experiment (A) and after intramammary administration of the bacteriophage preparation on day 14 of the experiment (B).

FIG. 7 shows the influence of antibiotics and phage preparation on the somatic cell count in milk in the udder quarters infected with E. coli and S. aureus.

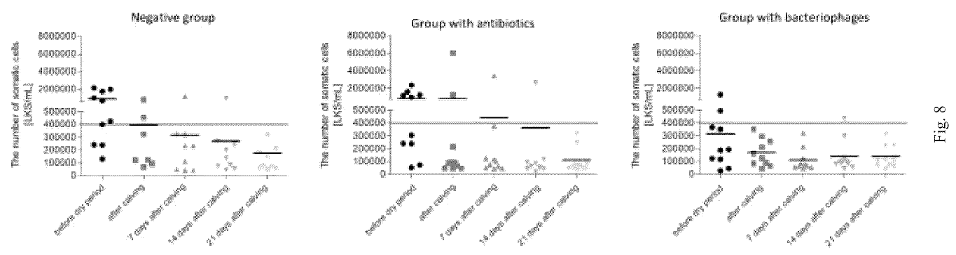

FIG. 8 shows the assessed somatic cell count in the milk in all tested quarters.

This description was completed with the following examples aimed to illustrate the reference invention better. The examples shall not be regarded as a full scope of the invention.

### EXAMPLE 1. BACTERIOPHAGE ISOLATION AND CHARACTERISTICS

### Isolation of Bacteriophages Active Towards Selected S. Aureus and E. Coli Strains From Environmental Samples

A unique collection of 37 different strains pathogenic for dairy cows was used for bacteriophage isolation, including 15 S. aureus strains, 1 Staphylococcus haemolyticus strain, 1 Staphylococcus chromogenes strain, 2 Staphylococcus CNS (coagulase-negative staphylococci) strains and 18 E. coli strains isolated from livestock with mastitis symptoms. The collection is the property of Proteon Pharmaceuticals S.A. All the strains were verified biochemically and genetically. The strains' diversification was confirmed with the PCR-MP and/or MLVF type PCR method. The strains were analysed for the presence of resistance genes to common antibiotics. The drug sensitivity was additionally tested with a disk diffusion and MIC method, according to CLSI (Clinical Laboratory Standards Institute) recommendations.

The bacteriophages were isolated from the samples of effluents, milk, and water: from udder washing, from the drinking lines and ponds, with a double-layer plate method and phage-particle enrichment. 34 bacteriophages specific to E. coli and 32 bacteriophages specific to S. aureus were isolated. To obtain purified bacteriophage strains, the phages were subjected to at least 5-times passaging from a single plaque on a solid medium. In order to select the bacteriophage preparation potential components, the isolated phages were subjected to characteristics involving: bacteriophage differentiation with an RFLP (Restriction Fragment Length Polymorphism) analysis, a test of phage specificity to E. coli and S. aureus strains isolated from animals with mastitis, assessment of the phage's lytic activity, bioinformatic analysis of the phages' genome sequence to determine their similarity, taxonomy, virulence and morphology assessment with an electron microscopy.

### Bacteriophage Differentiation With the RFLP Method

The isolated bacteriophages were subjected to genetic material isolation with the modified method presented by Su M. T. et al. [1998], followed by the RFLP analysis of the bacteriophages, which revealed 28 different strains specific to E. coli and 23 different strains specific to S. aureus.

### Testing Bacteriophage Specificity (Host Range)

The next stage involved determining with a spot test method of the specificity spectrum (host range) for the 51 isolated bacteriophages to 18 E. coli strains and 15 S. aureus strains isolated from animals with mastitis. It was demonstrated that among the tested bacteriophages, 10 specific to E. coli and 11 specific to S. aureus were characterised by a broad host range (lysis of bacterial lawn>50% of the tested strains), while 3 anti-E. coli phages were characterised by specificity supplementing the phages' specificity coverage to the collection of the strains. The genetic material of the bacteriophages with the desired specificity spectrum was subjected to sequencing. Table 1 summarises the results of the specificity analysis of 27 phages where the genomes were subjected to sequencing and subsequent genetic analysis.

TABLE 1
Specificity of selected bacteriophages to E. coli and S. aureus strains.

| Bacteriophage | | | | | | | | | | | | | | | | | | |
|---|---|---|---|---|---|---|---|---|---|---|---|---|---|---|---|---|---|---|
| specific to | E. coli strain | | | | | | | | | | | | | | | | | |
| E. coli | 90 | 91 | 92 | 93 | 94 | 95 | 96 | 97 | 98 | 99 | 100 | 101 | 103 | 104 | 117 | 118 | 132 | 133 |
| 241Ecol114PP | IL | NL | NL | NL | NL | IL | NL | NL | NL | NL | NL | NL | NL | IL | NL | CL | NL | IL |
| 303Ecol095PP | CL | NL | CL | CL | IL | CL | IL | CL | CL | CL | IL | IL | NL | NL | IL | NL | CL | CL |
| 308Ecol101PP | IL | IL | IL | CL | IL | CL | NL | CL | CL | IL | NL | CL | CL | IL | NL | NL | CL | CL |
| 310Ecol104PP | CL | IL | NL | CL | CL | CL | IL | CL | IL | CL | IL | CL | CL | CL | NL | CL | CL | CL |
| 348Ecol098PP | CL | NL | NL | NL | CL | CL | IL | CL | CL | CL | IL | CL | CL | NL | CL | NL | IL | IL |
| 306Ecol101PP | IL | NL | NL | CL | NL | CL | NL | CL | CL | CL | NL | IL | NL | NL | NL | NL | CL | CL |
| 307Ecol101PP | NL | IL | NL | IL | CL | IL | NL | CL | CL | IL | IL | CL | IL | CL | IL | IL | CL | CL |
| 309Ecol096PP | CL | CL | CL | CL | IL | IL | IL | NL | CL | IL | IL | IL | CL | IL | IL | CL | IL | CL |
| 318Ecol100PP | NL | NL | NL | NL | NL | NL | NL | NL | NL | NL | CL | NL | NL | NL | NL | NL | NL | NL |
| 319Ecol090PP | IL | NL | NL | IL | NL | NL | NL | NL | NL | NL | NL | NL | NL | NL | NL | NL | NL | NL |
| 320Ecol095PP | IL | NL | IL | IL | NL | CL | NL | NL | NL | IL | NL | IL | NL | NL | IL | IL | IL | IL |
| 322Ecol100PP | NL | NL | NL | NL | NL | NL | NL | NL | NL | NL | CL | NL | NL | NL | NL | NL | NL | NL |
| 324Ecol095PP | NL | NL | NL | NL | NL | CL | NL | NL | NL | IL | NL | NL | NL | NL | NL | NL | IL | IL |
| 325Ecol133PP | NL | NL | NL | NL | NL | NL | NL | NL | NL | IL | NL | NL | NL | NL | NL | NL | IL | IL |
| 346Ecol118PP | NL | NL | IL | CL | IL | CL | NL | IL | IL | CL | IL | NL | NL | IL | IL | CL | IL | IL |
| 347Ecol118PP | NL | NL | IL | CL | IL | CL | NL | NL | NL | CL | IL | NL | NL | IL | IL | CL | IL | IL |

| Bacteriophage | | | | | | | | | | | | | | | |
|---|---|---|---|---|---|---|---|---|---|---|---|---|---|---|---|
| Bacteriophage | | | | | | | | | | | | | | | |
| specific to | S. aureus strain | | | | | | | | | | | | | | |
| S. aureus | 58 | 59 | 60 | 62 | 63 | 67 | 69 | 75 | 76 | 79 | 80 | 82 | 83 | 90 | 91 |
| 351Saur083PP | IL | IL | IL | IL | IL | IL | IL | IL | CL | IL | IL | CL | CL | CL | CL |
| 355Saur083PP | IL | IL | IL | IL | IL | IL | IL | IL | IL | IL | IL | IL | IL | NL | NL |
| 357Saur119PP | CL | CL | CL | IL | IL | CL | IL | IL | IL | IL | IL | IL | IL | CL | IL |
| 350Saur075PP | IL | IL | IL | CL | IL | IL | IL | CL | IL | CL | IL | IL | IL | IL | IL |
| 356Saur083PP | IL | IL | IL | IL | IL | IL | IL | IL | IL | IL | IL | IL | IL | NL | IL |
| 339Saur067PP | IL | IL | IL | IL | CL | CL | IL | IL | IL | IL | IL | IL | IL | IL | IL |
| 341Saur063PP | CL | NL | IL | IL | NL | CL | CL | IL | IL | IL | IL | IL | IL | IL | IL |
| 311Saur063PP | IL | IL | IL | IL | CL | IL | IL | IL | IL | IL | IL | IL | IL | IL | IL |
| 312Saur075PP | CL | IL | IL | CL | CL | IL | CL | CL | IL | NL | IL | IL | IL | IL | IL |
| 316Saur075PP | CL | IL | IL | CL | CL | IL | CL | CL | IL | CL | IL | IL | IL | IL | IL |
| 333Saur075PP | CL | IL | IL | CL | CL | IL | CL | CL | IL | CL | IL | IL | IL | IL | IL |

Legend:
NL No lysis
CL Complete lysis
IL Incomplete lysis

### Genetic Characteristics of Bacteriophages

The DNA of selected phages (Table 1) was sequenced with the NGS (Next Generation Sequencing) method on the Illumina platform. The results were submitted de novo (SPAdes 3.11.1) and manually processed (FA_TOOL; UŁ), and the obtained sequences were annotated (DNA Master). Next, bioinformatic analysis was carried out to determine the replication cycle of bacteriophage.

It was discovered that among the 27 analysed bacteriophage strains, 10 performs a lytic cycle only (6 anti-E. coli and 4 anti-S. aureus phages). The phages were considered virulent because no genes responsible for lysogeny were found in their genomes.

### Testing the Lytic Activity

The lytic activity of 10 virulent bacteriophages was tested for all 37 E. coli and S. aureus strains from the collection of strains pathogenic for dairy cows. The 100 μL of 100-fold diluted ca. 20-hour bacterial culture was applied to four wells in a 96-well plate. Two wells with the applied bacterial culture were a positive control, while 20 μL of the given bacteriophage (test sample) with the count of 2×10⁸ PFU/well were placed in the other two wells. Medium (100 μL) was placed in the other two wells, while the next two wells included medium (100 μL) and bacteriophage lysate (20 μL) with the count of 2×10⁸ PFU/well (as negative controls). Then the plates were placed in a TECAN SUNRISE reader, and the samples' absorbance (OD₆₂₀) was measured every 20 minutes for 280 minutes of incubation at 37° C.

FIG. 1 shows sample results of lytic activity tests for 10 virulent bacteriophages, carried out on E. coli_133 and S. aureus_083 strains.

The 355Saur083PP, 356Saur083PP and 357Saur119PP bacteriophages strongly inhibited the growth of at least 90% of the tested S. aureus strains, while the 351Saur083PP bacteriophage strongly inhibited the growth of 27% of the strains. The 307Ecol101PP and 310Ecol104PP bacteriophages strongly inhibited the growth of at least 50% of E. coli strains. The 241Ecol014PP, 308Ecol101PP and 348Ecol098PP bacteriophages strongly inhibited the growth of at least 30% of the test strains. For the 303Ecol101PP bacteriophage, a strong inhibition of growth was observed for 28% of the strains.

Based on the results of bacteriophage specificity, lytic activity, assessment of the phages' replication cycle and their taxonomic diversity the following bacteriophages were selected as the phage cocktail components: 303Ecol101PP, 308Ecol101PP, 310Ecol104PP, 348Ecol098PP, 241Ecol014PP, 351Saur083PP, 355Saur083PP and 357Saur119PP.

### Taxonomy of the Phages Included in the Bacteriophage Preparation

Complete genetic characteristics were carried out for the 303Ecol101PP, 308Ecol101PP, 310Ecol104PP, 348Eco1098PP, 241Ecol014PP, 351Saur083PP, 355Saur083PP and 357Saur119PP bacteriophages being the bacteriophage preparation components to determine their similarity to the reference phages (Table 2, Table 3).

TABLE 2
Characteristics of phages specific to E. coli, included in the bacteriophage preparation.

| | | Bacteriophage | | | | |
|---|---|---|---|---|---|---|
| Feature | | 303Ecoll01PP | 308Ecoll01PP | 310Ecoll04PP | 348Ecol098PP | 241Ecol014PP |
| Host: E. coli | | Ecol_095PP2 | Ecol_098PP201 | Ecol_104PP201 | Ecol_098PP20 | Ecol_242PP2017 |
| Morphology | | icosahedral capsid and a long contraktail tail | | | | |
| Genome size [bp] | | 166901 | 169543 | 167023 | 170844 | 138401 |
| ORF | | 272 | 276 | 269 | 270 | 215 |
| tRNA | | 11 | 2 | 10 | 3 | 5 |
| Content of GC pairs [%] | | 35.4 | 37.7 | 35.6 | 37.5 | 43.6 |
| Taxonomy of | Order | Caudovirales | Caudovirales | Caudovirales | Caudovirales | Caudovirales |
| the reference | Family | Myoviridae | Myoviridae | Myoviridae | Myoviridae | Myoviridae |
| strain | Subfamily | Teveuvirinae | Teveuvirinae | Teveuvirinae | Teveuvirinae | Vequmtavirinae |
| | Genus | Tequatrovinis | Masigvinis | Tequatrovirus | Masigvinis | Vequintavirus |
| | Species | Escherichia | Escherichia | Enterobacteria | Shigella phage | Escherichia |
| | | phage teqdroes | phage | phage Kha5h | SSE1 | phage slur 12 |
| | | | vB_EcoM_JS09 | | | |

| | | | | | |
|---|---|---|---|---|---|
| Analysed phage's genome | 97% | 98% | 96% | 95% | 95% |
| coverage to the reference | | | | | |
| genome [%] | | | | | |
| Similarity of the analysed | 98% | 98% | 97% | 99% | 96% |
| phage's genome to the | | | | | |
| reference genome [%] | | | | | |

TABLE 3
Characteristics of phages specific to S. aureus, included in the bacteriophage preparation.

| | | Bacteriophage | | |
|---|---|---|---|---|
| Feature | | 351Saur083PP | 355Saur083PP | 357Saur119PP |
| Host: S. aureus | | S. aureus_083PP2016 | S. aureus _083PP2016 | S. aureus _141PP2018 |
| Morphology | | icosahedral capsid | icosahedral capsid | icosahedral capside |
| | | and a short | and a long | and a long |
| | | non-contraktail tail | contraktail tail | contraktail tail |
| Genome size [bp] | | 17209 | 143709 | 140580 |
| ORF | | 19 | 222 | 215 |
| tRNA | | 0 | 4 | 4 |
| Content of GC pairs [%] | | 29.3 | 30.4 | 30.4 |
| Taxonomy of | Order | Caudovirales | Caudovirales | Caudovirales |
| the reference | Family | Podoviridae | Herelleviridae | Herelleviridae |
| strain | Subfamily | Rakietenvirinae | Twortvirinae | Twortvirinae |
| | Genus | Rosenblumvirus | Kavvirus | Kavvirus |
| | Type | Staphylococcus phage | Staphylococcus phage | Staphylococcus phage |
| | | GRCS | vB_SauM_LM12 | Stab21 |

| | | | |
|---|---|---|---|
| Analysed phage's genome | 99% | 94% | 92% |
| coverage to the reference | | | |
| genome [%] | | | |
| Similarity of the analysed | 97% | 99% | 99% |
| phage's genome to the | | | |
| reference genome [%] | | | |

The bacteriophage's morphology was evaluated with the JEOL 1010 TEM transmission electron microscope (FIG. 2). After washing three times and centrifuging (15,000 rpm) for three hours, the phages were suspended in a 5% ammonium molybdate solution. Then the suspensions were applied to formware-coated and carbon-sprayed copper meshes and contrasted for 45 s with 2% phosphovolframic acid in darkness. The photos of the phages were taken in the Laboratory of Microscopic Imaging and Specialised Biological Techniques, Faculty of Biology and Environmental Protection, University of Łódź.

### Testing Sensitivity of the Variants Resistant to Selected Bacteriophages

In order to verify the optimum composition of a phage preparation, single phages' lytic activity towards bacteria was analysed if variants insensitive to individual phage components emerged. Seven host strains were used in the tests for which mutants resistant to selected phages included in the developed cocktail were searched.

100 μL of each of the following bacteriophages were added to each Eppendorf tube: 303Ecol101PP, 308Ecol101PP, 348Ecol098PP, 241Ecol014PP, 310Ecol104PP, 351Saur083PP, 355Saur083PP, and 357Saur119PP with 1×10⁹ PFU/mL count. Then 100 μL of 100-times diluted bacterial culture with the density of OD₆₀₀=0.5 (ca. 10⁶ CFU/mL) were added to each tube. Simultaneously, a bacteria control sample was prepared, containing 100 μL of the growth medium and 100 μL of 100-times diluted bacterial culture. The samples were incubated for 10 minutes at 37° C. After incubation, 100 μL of the sample were collected, and culture was made with a glass spreader on plates with adequately prepared growth medium. The plates for testing the bacteria variants resistant to individual phages were prepared by pouring the top agar containing 100 μL of the bacteriophage suspension onto an agar-solidified medium. The plates intended for culturing the bacteria from the control sample were prepared by pouring out the top agar containing 100 μL of the solution in which the bacteriophages were suspended onto an agar-solidified medium. The plates were incubated for 24 hours at 37° C. The bacteria colonies, which grew on the plates with single phages added, were re-cultured onto a solid and liquid medium and then their resistance was verified with a spot test method and by standardising the suspension of the bacteriophage to which they became resistant. The resistant variants prepared this way were banked in the collection of strains, and the spot test method was used to check if the other bacteriophages included in the developed cocktail remained active towards them. Table 4 summarises the experiment results.

TABLE 4
Specificity of the bacteriophages included in the developed cocktail to phage-resistant bacteria variants.

| | Strain used to isolate phage resistant variants | | | | | | | | | | | | | | |
|---|---|---|---|---|---|---|---|---|---|---|---|---|---|---|---|
| | E. coli | | | | | | | | | | | | | | |
| | 101 | E. coli 095 | | | E. coli 095 | | | E. coli 098 | | | | E. coli 104 | | | |
| Bacteriophage | Bacteriophage | | | | | | | | | | | | | | |
| used for | 303Ecol101PP | | | | 308Ecol101PP | | | 348Ecol098PP | | | | 241Ecol014PP | | | |
| sensitivity | Resistant variants obtained | | | | | | | | | | | | | | |
| testing | 227 | 228 | 229 | 230 | 235 | 236 | 237 | 241 | 242 | 243 | 244 | 261 | 262 | 263 | 264 |
| 303Ecol101PP | − | − | − | − | + | + | + | − | − | − | − | − | − | − | − |
| 308Ecol101PP | + | + | + | − | − | − | − | + | − | − | − | + | + | + | − |
| 310Ecol104PP | + | + | + | + | + | + | + | + | − | − | − | − | − | − | − |
| 348Ecol098PP | + | + | + | + | + | + | + | − | − | − | − | + | − | − | − |
| 241Ecol014PP | − | − | − | + | + | + | + | + | + | + | + | − | − | − | − |
| 351Saur083PP | NT | NT | NT | NT | NT | NT | NT | NT | NT | NT | NT | NT | NT | NT | NT |
| 355Saur083PP | NT | NT | NT | NT | NT | NT | NT | NT | NT | NT | NT | NT | NT | NT | NT |
| 357Saur119PP | NT | NT | NT | NT | NT | NT | NT | NT | NT | NT | NT | NT | NT | NT | NT |

| | Strain used to isolate phage resistant variants | | | | | | | | | | | |
|---|---|---|---|---|---|---|---|---|---|---|---|---|
| | S. aureus 83 | | | | | | | | | | | |
| | Bacteriophage | | | | | | | | | | | |
| | 310Ecol104PP | | | 351Saur083PP | | | | 355Saur083PP | | | | 357Saur119PP |
| Bacteriophage | Resistant variants obtained | | | | | | | | | | | |
| used for | | | | | | | | | | | | resistant |
| sensitivity | | | | | | | | | | | | variants were |
| testing | 265 | 266 | 300 | 119 | 120 | 121 | 122 | 123 | 124 | 125 | 126 | not obtained |
| 303Ecol101PP | − | − | − | NT | NT | NT | NT | NT | NT | NT | NT | NT |
| 308Ecol101PP | + | + | + | NT | NT | NT | NT | NT | NT | NT | NT | NT |
| 310Ecol104PP | − | − | − | NT | NT | NT | NT | NT | NT | NT | NT | NT |
| 348Ecol098PP | − | − | − | NT | NT | NT | NT | NT | NT | NT | NT | NT |
| 241Ecol014PP | + | + | + | NT | NT | NT | NT | NT | NT | NT | NT | NT |
| 351Saur083PP | NT | NT | NT | − | − | − | − | − | − | − | − | NT |
| 355Saur083PP | NT | NT | NT | − | − | − | − | − | − | − | − | NT |
| 357Saur119PP | NT | NT | NT | + | + | + | + | + | + | + | + | NT |

Legend:
− No effect
+ Lysis
NT Not tested

The 357Saur119PP bacteriophage was demonstrated not to cause the onset of phage-resistance in S. aureus bacteria and reveals specificity to phage-resistant bacteria variants obtained after induction with other S. aureus bacteriophages. Seventeen (17) of the 18 obtained phage-resistant E. coli variants remain sensitive to at least two other bacteriophages included in the cocktail. The test results confirm the optimum composition of the bacteriophage cocktail that prevents the emergence of bacteria variants insensitive to individual phages after using the preparation.

### EXAMPLE 2. EVALUATION OF THE DEVELOPED BACTERIOPHAGE COCKTAIL'S EFFICACY TO PREVENT THE FORMATION AND CONTROL THE BIOFILM

An efficacy test of the developed bacteriophage cocktail was carried out for the destruction of 24-hour biofilm and prevention of its formation for 22 E. coli strains (18 strains from the mastitis-causing strain collection and 4 resistant variants) and 18 S. aureus strains (15 strains from the mastitis-causing strains and 3 resistant variants). In the biofilm destruction test, 100 μL of 100-times diluted overnight bacterial culture were applied to a 96-well plate and incubated for 24 hours at 37° C. in a humid chamber. Then the suspensions were removed from above the well bottom, the biofilms were washed with saline, and 100 μL of the bacteriophage cocktail with the count of 2×10⁸ PFU/mL were added to the wells. The medium used for the cocktail preparation was added to the control wells. The plate was re-incubated for 24 hours at 37° C. in a humid chamber, and then an MTT assay was carried out where yellow tetrazolium salt (3-(4,5dimethylthazol-2-yl)-2,5-diphenyltetrazolium bromide) exposed to dehydrogenases in living cells is reduced to purple formazan crystals, which affects the absorbance value (OD₅₇₀): the higher the formazan concentration, the higher the number of living bacteria adsorbed to the plate sample is. The same procedure was applied in the biofilm formation prevention test, whereby bacterial suspension and bacteriophage cocktail were simultaneously applied to the plate.

The tests revealed that the developed cocktail inhibited min. 99% of biofilm formation for 100% of the tested S. aureus strains (18/18) and destroyed min. 50% of the biofilm for 39% of the strains (7/18) and min. 20% of the biofilm for 67% strains (12/18) (FIG. 3.A).

For E. coli strains, the developed cocktail inhibited min. 50% of the biofilm formation for 50% of the tested E. coli strains (11/22) and destroyed min. 50% of the biofilm for 59% of the strains (13/22) and min. 20% of the biofilm for 86% of the strains (19/22) (FIG. 3.B).

During biofilm formation prevention testing, optical density in time was measured at the λ=600 nm (OD₆₀₀) wavelength. The bacteria growth curves for sample E. coli_133 and S. aureus_083 strains in the presence of bacteriophage cocktail and in a control system with no bacteriophages demonstrate high activity in inhibiting the bacteria growth (FIG. 4).

### EXAMPLE 3. FORMULATION OF A BACTERIOPHAGE PREPARATION IN THE FORM OF GEL, ACCORDING TO THE INVENTION

The preparation being the subject of the invention has a form of gel obtained by mixing an iota-carrageenan solution with the developed bacteriophage cocktail. An aqueous solution of iota-carrageenan with a concentration ranging from 0.4 to 8.0% w/v is prepared in the first stage. To that end, an iota-carrageenan fraction is used that form gels in which no syneresis occurs. In the selected iota-carrageenan concentration range, gels are prepared with the cross-linking density enabling their combination with a bacteriophage mixture at the desired temperature. The iota-carrageenan solution is then sterilised and cooled to room temperature. A phage cocktail with the 4×10⁸ PFU/mL count (equivalent mixture of components specific to E. coli and S. aureus) in a 1:1 volumetric ratio. Both ingredients are mixed in sterile conditions at 36° C., ensuring the preparation ingredients' stability. Such a preparation is stored at a refrigeration temperature.

In order to determine the gel's optimum composition, a number of experiments were performed, testing the addition of ions affecting polymer cross-linking, such as KCl and CaCl₂. Sample tested systems are shown in Table 5.

TABLE 5
Examples of gel options tested during gel composition optimisation.

| | Iota- | | | |
|---|---|---|---|---|
| | carrageenan | Concentration | Concentration | |
| Tested option | concentration | KCl | CaCl₂ | Characteristic |
| number | [%] | [%] | [%] | features |
| 1 | 3.19 | 2.16 | — | Strongly cross-linked, difficulty occurs |
| | | | | during mixing with a phage cocktail. |
| | | | | Syneresis occurs during storage. |
| 2 | 2.77 | — | 0.42 | Strongly cross-linked, difficulty occurs |
| | | | | during mixing with a phage cocktail. |
| | | | | No syneresis during storage. |
| 3 | 2.77 | 4.13 | — | Mixing with the phage cocktail occurs |
| | | | | to the correct extent. |
| | | | | Syneresis cannot be observed with a |
| | | | | naked eye, but the gel loses its |
| | | | | homogeneity during storage. |

It was determined that because of the intended use and administration route of the preparation, the carrier meant for manufacturing a bacteriophage preparation for mastitis treatment should maintain the phages' adequate activity as well as specific homogeneity and durability.

Only the carrier according to the invention, with the composition summarised in Table 6, fulfilled all the presented requirements among the tested systems.

TABLE 6
Gel composition developed according to the invention.

| Iota-carrageenan | Concentration | Concentration | |
|---|---|---|---|
| concentration | KCl | CaCl₂ | Characteristic |
| range [%] | [%] | [%] | features |
| 0.40-8.00 | — | — | Mixing with the phage cocktail |
| | | | occurs to the correct extent. |
| | | | Syneresis does not occur during storage. |
| | | | Maintaining the bacteriophage's |
| | | | adequate stability. |

The following problems were observed during the tests for the other compositions:
too strong cross-linking of the gel preventing its thorough mixing with the bacteriophage cocktail at 36° C.,
syneresis occurrence during storage,
for cross-linking ions—a risk that the change in the ion composition in the buffer where the bacteriophages are suspended reduces their stability.

### EXAMPLE 4. EFFICACY EVALUATION OF THE PREPARATION ACCORDING TO THE INVENTION TO PREVENT BACTERIAL BIOFILM FORMATION

Bacterial cultures of E. coli_133 and S. aureus_083 strains, prepared by 25-times dilution of the overnight culture were applied to a 96-well plate in the amount of 50 μL per well, and then an equal volume of the preparation according to the invention, with the count of 2×10⁸ PFU/mL was added to the wells. Polymer gel free of phages was added to the control wells containing relevant bacterial cultures. The plate was incubated for 24 h at 37° C. in a humid chamber, and then an MTT assay was carried out in the same way as described in Example 2.

The tests revealed that the preparation according to the invention, demonstrated efficacy to prevent bacterial biofilm formation amounting to 46.8±18.2% for E. coli_133 and 94.2±2.1% for S. aureus_083.

### EXAMPLE 5. EFFICACY OF THE PREPARATION ACCORDING TO THE INVENTION IN MILK

In order to evaluate the efficacy of the preparation according to the invention, an in vitro test was carried out on a milk model that highly reflects the conditions in a dairy cattle's udder owing to the content of proteins, amino acids, microelements, vitamins, lactose and other ingredients. Milk containing 3.2% of fat, pasteurised at a low temperature, was centrifuged (4,500 rpm, 4° C., 10 min) in Falcon type centrifuge tubes (50 mL) and the separated cream layer was removed. E. coli_133 and S. aureus_083 strain cultures with OD₆₀₀=1 were adequately diluted in saline to obtain suspensions with 1×10⁵ CFU/mL. 2 mL of adequate bacterial suspensions were added to 18 mL of milk (to obtain the density of 1×10⁴ CFU/mL). The mixture was divided into 9 mL portions and placed in 2 sterile Falcon tubes (50 mL); then 1 mL of the preparation according to the invention (tested system) and 1 mL of gel free of bacteriophages (control system) were added to the tubes. After 24 h of the samples' incubation at 37° C., including shaking (140 rpm), the bacteria density was determined for each system with a serial dilution method, using MacConkey (for E. coli) or Chapman (for S. aureus) medium. The preparation's efficacy was expressed as a percentage determining the bacteria count decrease in the test sample compared to the bacteria count in the control sample assumed as 100%. It was demonstrated that the applied preparation according to the invention inhibited the E. coli and S. aureus bacteria growth in milk by 21.6±3.8% and 30.6±9%, respectively.

### EXAMPLE 6. EFFICACY EVALUATION OF THE PREPARATION ACCORDING TO THE INVENTION TO PREVENT BACTERIAL BIOFILM FORMATION BASED ON A COLLAGEN MATRIX MODEL

In order to evaluate the bacteriophage preparation's efficacy, a test was carried out on a collagen matrix model, which reflects the in vivo conditions in the udder tissue for the presence of specific proteins, owing to the use of fetal calf serum. With this purpose in mind, collagen matrices (250 μL) were prepared in 2 mL round-bottom tubes according to the method described by Werthen M. et al. [2010] as well as overnight cultures of E. coli_133 and S. aureus_083 strains incubated at 37° C., with shaking at 140 rpm. 100 μL of phage preparation with the 2×10⁸ PFU/mL count, heated for an hour at 37° C., were applied to the prepared matrices. Bacteriophage-free gel treated in the same way as the phage preparation was the control. Then, 250 μL of bacterial inoculum diluted in saline to the density of ˜1×10⁴ CFU/mL were added to each sample and incubated at 37° C. After 24 hours of incubation, the liquefied suspension was collected from above the matrix, and the matrix was rinsed three times with 0.5 mL of PBS solution. After rinsing, the matrix was liquefied. To that end, 250 μL of collagenase solution at 1 mg/mL concentration was applied to the matrix and incubated for 3-3.5 h at 37° C. Then, bacteria density was identified as CFU/mL. The test was repeated at least three times.

It was demonstrated that the bacteriophage preparation according to the invention, tested on a collagen matrix model representing in vivo conditions, reduced the growth of E. coli bacteria by 30.3±19.1% and S. aureus bacteria by 49.3±13.8%.

### EXAMPLE 7. TESTING STORAGE STABILITY OF THE PREPARATION ACCORDING TO THE INVENTION

The preparation's storage stability was tested at 4° C. for 12 months in a liquid bacteriophage cocktail and a gel preparation with the 2×10⁸ PFU count per millilitre or gram, respectively. The cocktail and the gel demonstrated at least 95% stability after 12 months of storage at 4° C. (Table 7).

TABLE 7
Storage stability of the bacteriophage preparation
in a liquid and gel form (4° C.).
Preparation's storage stability at 4° C.[log %]

Formulation

| | Bacteriophage | Gel with a |
|---|---|---|
| Time [month] | cocktail | bacteriophage |
| 0 | 100 | 100 |
| 3 | 97 | 99 |
| 6 | 97 | 97 |
| 9 | 98 | 98 |
| 12 | 95 | 95 |

### EXAMPLE 8. EFFICACY EVALUATION OF USING BACTERIOPHAGE PREPARATION ACCORDING TO THE INVENTION ADMINISTERED INTRAMAMMARY TO DAIRY COWS WITH MASTITIS CAUSED BY E. COLI OR S. AUREUS

A preparation containing eight bacteriophage components manufactured on a semi-technical scale, with a total count of 2×10⁸ PFU/g was used for the tests. The obtained preparation did not reveal any presence of microorganisms, which was confirmed in microbiological purity tests.

The test covered 30 dairy cows during lactation, in which mastitis caused by E. coli or S. aureus bacteria was detected and microbiologically confirmed in at least one udder quarter, at a simultaneous lack of infection with other mastitis-causing bacteria. The animals were randomly divided into 3 equal groups:
A group taking antibiotics—cows with spontaneous mastitis, which received intramammary antibiotics (amoxicillin with clavulanic acid) every 12 hours for 2.5 days (5 antibiotics dosed).
Negative group—cows with spontaneous mastitis, not getting either a bacteriophage preparation or antibiotics.
Group getting bacteriophages—cows with spontaneous mastitis, receiving intramammary 5 mL of bacteriophage preparation with 2×10⁸ PFU/g count every 12 h for 3.5 after milking (7 preparation doses).

Each group included 5 cows with E. coli infection and 5 cows with S. aureus infection.

The following parameters were monitored during the entire experiment period (day 0-14 of the experiment):
number of infected udder quarters,
the cows' general condition based on the treatment summary report,
evaluation of mastitis symptoms occurrence in each udder quarter,
somatic cell count (SCC) in milk in all udder quarters.

The results obtained during the influence evaluation of antibiotics and bacteriophage preparation on the number of infected udder quarters are shown in FIG. 5.

Based on the results, for E. coli infection (FIG. 5.A), it was demonstrated that in the group receiving intramammary preparation according to the invention, no infection symptoms were observed on days 4 and 7 of the experiment, while on day 14 of the experiment, the bacteria were detected only in one quarter. The cows can have been re-infected with E. coli bacteria. For S. aureus infection (FIG. 5.B), the highest short-term efficacy (nearly 90% decrease) was observed in the group of cows receiving antibiotics on day 4 of the experiment. The bacteriophages caused a 40% infection decrease on day 4, but on day 14, up to 80% increase in the infection was observed in the tested quarters.

The influence of the antibiotics and bacteriophage preparation on the mastitis intensity in different quarters included the evaluation of the following:
1. asymmetry (oedema) based on the comparison of the quarter's condition in A-C and B-D pairs,
2. pain—based on the udder's palpation and observation of defensive reactions such as kicking, avoiding the touch, etc.,
3. skin redness—based on comparing the colour of the examined quarter's skin colour with the skin colour on the rest of the udder,
4. macroscopic changes in the milk, including changes in the milk's consistency, colour or odour,
5. occurrence of general symptoms, including but not limited to apathy, loss of appetite, fever and lying behaviour,
6. occurrence of other symptoms, including diarrhoea, lameness, blue udder skin, foul secretion from the udder, gas released from the udder and death.

The results of the influence of the preparation according to the invention are summarised in Table 8 and FIG. 6.

TABLE 8
Bacteriophage preparation's influence on the occurrence of clinical
symptoms in the infected udder quarters in dairy cows with mastitis.
The frequency of the tested mastitis symptoms [% of the tested animals]

| | | Tested group | | | | | |
|---|---|---|---|---|---|---|---|
| | | Group | | | | Group | |
| | | receiving | | Negative | | receving | |
| | | antibiotics | | group | | bacteriophages | |
| Etiological | | Day of experiment | | | | | |
| factor | Symptoms | 0 | 14 | 0 | 14 | 0 | 14 |
| E. coli | Asymmetry | 100 | 20 | 100 | 40 | 100 | 0 |
| | Pain | 80 | 0 | 80 | 20 | 100 | 0 |
| | Redness | 20 | 0 | 0 | 0 | 0 | 0 |
| | Changes in | 100 | 40 | 100 | 40 | 100 | 0 |
| | the milk | | | | | | |
| | General symptoms | 80 | 20 | 80 | 20 | 100 | 0 |
| | Other | 20 | 0 | 20 | 0 | 20 | 0 |
| S. aureus | Asymmetry | 50 | 13 | 40 | 40 | 0 | 0 |
| | Pain | 13 | 0 | 20 | 0 | 0 | 0 |
| | Redness | 0 | 0 | 0 | 0 | 0 | 0 |
| | Changes in | 50 | 25 | 60 | 60 | 80 | 20 |
| | the milk | | | | | | |
| | General symptoms | 0 | 0 | 0 | 0 | 0 | 0 |
| | Other | 0 | 0 | 0 | 0 | 0 | 0 |

Based on the results, it was demonstrated that on day 14 of the experiment, as a result of intramammary administration of the bacteriophage preparation, improvement in the milk quality and alleviation of mastitis symptoms in the infected udder quarters were observed. Most analysed mastitis symptoms resolved entirely on day 14 of the experiment, both in the quarters infected with E. coli and S. aureus compared to experiment day 0. Only in the case of S. aureus infection, the changes in milk remained in 20% of the tested animals undergoing phage therapy. Complete resolution of symptoms (oedema, lower milk quality, etc.) was observed only in the group receiving the bacteriophage preparation, contrary to the group which received antibiotics.

It was observed that in the case of infection caused by E. coli (FIG. 7), the somatic cell count (SCC) in milk was noted in each tested group, whereby the lowest SCC was observed on day 14 of the experiment. In the case of S. aureus infection (FIG. 7), the most effective SCC reduction was observed in the group which received bacteriophages on day 14 of the experiment (below the critical value of 400,000 cells/mL), while in the group treated with antibiotics, the value was exceeded. Although spontaneous reduction was observed in the SCC value to the standard range, the decrease was much higher in the group that received the preparation according to the invention. In the group receiving the antibiotics on day 7 of the experiment, a significant reduction in the SCC occurred, while its increase was reported on day 14 of the experiment.

### EXAMPLE 9. EFFICACY EVALUATION OF USING BACTERIOPHAGE PREPARATION ACCORDING TO THE INVENTION, ADMINISTERED INTRAMAMMARY TO DAIRY COWS IN THE DRY PERIOD TO PREVENT MASTITIS CAUSED BY E. COLI OR S. AUREUS

A preparation containing eight bacteriophage components manufactured on a semi-technical scale, with a total count of 2×10⁸ PFU/g was used for the tests. The obtained preparation did not reveal any presence of microorganisms, which was confirmed in microbiological purity tests.

The study covered 30 healthy dairy cows in a dry period. The animals were randomly divided into 3 equal groups:
Negative group—healthy cows which received neither a bacteriophage preparation nor antibiotics after the last milking before the dry period,
Group receiving bacteriophages—healthy cows which received intramammary 10 mL of bacteriophage preparation with 2×10⁸ PFU/g count (1 preparation dose) after the last milking before the dry period,
Group receiving antibiotics—healthy cows which received intramammary antibiotics (benethamine penicillin with penethamate hydroiodide and framycetin sulphate) after the last milking and before the dry period (1 antibiotics dose).

Each group covered 10 healthy cows.

The following parameters were monitored during the experiment (dry period and 21 days after calving):
the cows' general condition based on the treatment summary report,
evaluation of mastitis symptoms occurrence in each udder quarter,
somatic cell count in milk in all udder quarters.

Based on the results (FIG. 8), it was demonstrated that the mean somatic cell count in 1 mL of milk in the group receiving intramammary preparation according to the invention was lower than 400,000 SCC/mL (permissible SCC in 1 mL of milk) in each study time point. For cows that received antibiotics, the mean somatic cell count reached the permissible level only 14 days after calving. Additionally, it was observed that the SCC in 1 mL of milk in the group receiving intramammary preparation according to the invention was lower than in the negative control group.

### LITERATURE

Schukken, Y. H., Gunther, J., Fitzpatrick, J., Fontaine, M. C., Goetze, L., Holst, O., Leigh, J., Petzl, W., Schuberth, H. J., Sipka, A., Smith, D. G. E., Quesnell, R, Watts, J., Yancey, R, Zerbe, H., Guijar, A., Zadoks, R. N., Seyfert, H. M. Host-response patterns of intramammary infections in dairy cows. Vet Immunol Immunopath. 2011.144: 270-289.
Kuipers, A., Koops, W. J., Wemmenhove, H. Antibiotic use in dairy herds in the Netherlands from 2005 to 2012. Journal of Dairy Science. 2016.99(2): 1632-1648.
Burmańczuk, A., Tomasz, G., Gbylik-Sikorska, M., Gajda, A., Kowalski, C. Withdrawal of Amoxicillin and Penicillin G Procaine from Milk after Intramammary Administration in Dairy Cows with Mastitis. J Vet Res. 2017, 61(1): 37-43.
Crispie, F., Flynn, J., Ross, R. P., Hill, C., Meaney, W. J. Dry cow therapy with a non-antibiotic intramammary teat seal—a review. Ir Vet J. 2004.57(7): 412-418.
Gomes, F., Saavedra, M. J., Henriques, M. Bovine mastitis disease/pathogenicity: evidence of the potential role of microbial biofilms. Pathog Dis. 2016,74(3): pii: ftw006.
Teale, C. J., David, G. P., Antibiotic resistance in mastitis bacteria. Proceedings of the British Mastitis Conference. 1999, 24-29.
Hillerton, J. E., Kliem, K. E. Effective treatment of Streptococcus uberis clinical mastitis to minimise the use of antibiotics. J Dairy Sei. 2002, 85(4): 1009-1014.
Vasudevan, P., Nair, M. K., Annamalai, T., Venkitanarayanan, K. S. Phenotypic and genotypic characterisation of bovine mastitis isolates of Staphylococcus aureus for biofilm formation. Vet Microbiol. 2003.92(1-2): 179-185.
Melchior, M. B., Vaarkamp, H., Fink-Gremmels, J. Biofilms: a role in recurrent mastitis infections? Vet J. 2006, 171(3): 398-407.
Łubowska, N., Piechowicz, L. Biofilm Staphylococcus aureus i rola bakteriofagów w jego eradykacji. Postępy Hig Med Dosw (online). 2018.72: 101-107.
Raza, A., Muhammad, G., Sharif, S., Atta, A. Biofilm Producing Staphylococcus aureus and Bovine Mastitis: A Review. Molecular Microbiology Research. 2013.3(1): 1-8.
Costa, J. C. M., de Freitas Espeschit, I., Pieri, F. A., Benjamin, L. A., Scatamburlo Moreira, M A. Increase in biofilm formation by Escherichia coli under conditions that mimic the mastitic mammary gland. Cienc. Rural. 2014.44(4): 666-671.
Matsuzaki, S., Yasuda, M, Nishikawa, H., Kuroda, M, Ujihara, T., Shuin, T., Shen, Y., Jin, Z., Fujimoto, S″ Nasimuzzaman, M. D., Wakiguchi, H., Sugihara, S., Sugiura, T., Koda, S., Muraoka, A., Imai, S. Experimental protection of mice against lethal Staphylococcus aureus infection by novel bacteriophage φMRI 1. J Infect Dis. 2003, 187: 613-624.
Yu, Y. P., Gong, T., Jost, G, Liu, W. H., Ye, D. Z., Luo, Z. H. Isolation and characterisation of five lytic bacteriophages infecting a Vibrio strain closely related to Vibrio owensii. FEMS Microbiol Lett. 2013.348: 112-119.
Elbreki, M., Ross, R. P., Hill, C., OMahony, J., Mcauliffe, O., Coffey, A. Bacteriophages and their derivatives as biotherapeutic agents in disease prevention and treatment. J Virol. 2014.2014: 1-20.
Golkar, Z., Bagasra, O., Pace, D. G. Bacteriophage therapy: a potential solution for the antibiotic resistance crisis. J Infect Dev Ctries. 2014.8: 129-136.
Li, Z., Li, X., Zhang, J., Wang, X., Wang, L., Cao, Z., Xu, Y. Use of phages to control Vibrio splendidus infection in the juvenile sea cucumber Aposlichopus japonicus. Fish Shellfish Immunol. 2016.54: 302-311.
Brüssow, H. Phage therapy: the Escherichia coli experience. Microbiology. 2005.151: 2133-2140.
Patent US20030235560A1
Zhang, Q., Xing, S., Sun, Q., Pei, G., Cheng, S., Liu, Y., An, X., Zhang, X., Qu, Y., and Tong, Y. Characterization and complete genome sequence analysis of a novel virulent Siphoviridae phage against Staphylococcus aureus isolated from bovine mastitis in Xinjiang, China. Virus Genes. 2017.53: 464-476.
García, P., Madera, C., Martínez, B., Rodríguez, A., and Evaristo Suárez, J. Prevalence of bacteriophages infecting Staphylococcus aureus in dairy samples and their potential as biocontrol agents. Journal of Dairy Science. 2009.92: 3019-3026.
Soo Son, J., Bae Kim, E., Jung Lee, S., Youn Jun, S., Jun Yoon, S., Hyeon Kang, S., and Jaie Choi, Y. Characterization of Staphylococcus aureus derived from bovine mastitis and isolation of two lytic bacteriophages. J. ul. Gen Appl. Microbiol. 2010.56: 347-353.
Zhang, L., Bao, H., Wei, C., Zhang, H., Zhou, Y., and Wang, R. Characterization and partial genomic analysis of a lytic Myoviridae bacteriophage against Staphylococcus aureus isolated from dairy cows with mastitis in Mid-east of China. Vims Genes. 2015.50: 111-117.
Li, L., Zhang, Z. Isolation and characterisation of a virulent bacteriophage SPW specific for Staphylococcus aureus isolated from bovine mastitis of lactating dairy cattle. Mol Biol Rep. 2014.41: 5829-5838.
Han, J. E., Kim, J. H., Hwang, S. Y., Choresca, C. H., Shin, S. P., Jun, J. W., Chai, J. Y., Park, Y. H., and Park, S. C. Isolation and characterisation of a Myoviridae bacteriophage against Staphylococcus aureus isolated from dairy cows with mastitis. Research in Veterinary Science. 2013.95: 758-763.
Dias, R. S., Eller, M. R., Duarte, V. S., Pereira, A. L., Silva, C. C., Mantovani, H. C., Oliveira, L. L., de A. M Silva, E., and De Paula, S. O. Use of phages against antibiotic-resistant Staphylococcus aureus isolated from bovine mastitis 1. Journal of Animal Science. 2013.91: 3930-3939.
Sangha, K. K., Kumar, B. V. S., Agrawal, R. K., Deka, D., and Verma, R. Proteomic Characterization of Lytic Bacteriophages of Staphylococcus aureus Isolated from Sewage Affluent of India. International Scholarly Research Notices. 2014.2014: 1-6.
Varela-Ortiz, D. F., Barboza-Corona, J. E., González-Marrero, J., León-Galván, M. F., Valencia-Posadas, M., Lechuga-Arana, A. A., Sánchez-Felipe, C. G., Ledezma-García, F., Gutiérrez-Chávez, A. J. Antibiotic susceptibility of Staphylococcus aureus isolated from subclinical bovine mastitis cases and in vitro efficacy of bacteriophage. Vet Res Commun. 2018, 42(3): 243-250.
Porter, J., Anderson, J., Carter, L., Donjacour, E., Paros, M. In vitro evaluation of a novel bacteriophage cocktail as a preventative for bovine coliform mastitis. J Dairy Sei. 2016.99(3): 2053-2062.
Horiuk, Y. V., Kukhtyn, M. D., Stravskyy, Y. S., Klymnyuk, S. I., Vergeles, K. M., Horiuk, V. V. Influence of staphylococcal Phage SAvB14 on biofilms, formed by Staphylococcus aureus variant bovis. Regulatory Mechanisms in Biosystems. 2019.10(3): 314-318.
Patent No. CA2661896
Kumaran, D., Taha, M, Yi, Q., Ramirez-Arcos, S., Diallo, J. S., Carli, A., Abdelbary, H. Does Treatment Order Matter? Investigating the Ability of Bacteriophage to Augment Antibiotic Activity against Staphylococcus aureus Biofilms. Front Microbiol. 2018.9: 127.
Tkhilaishvili, T., Lombardi, L., Klatt, A. B., Trampuz, A., Di Luca, M. Bacteriophage Sb-1 enhances antibiotic activity against biofilm, degrades exopolysaccharide matrix and targets persisters of Staphylococcus aureus. Int J Antimicrob Agents. 2018.52(6): 842-853.
Rahman, M., Kim, S., Kim, S. M., Seol, S. Y., Kim, J. Characterization of induced Staphylococcus aureus bacteriophage SAP-26 and its anti-biofilm activity with rifampicin. Biofouling. 2011.27: 1087-1093.
Ribeiro, K. V. G., Ribeiro, C., Dias, R. S., Cardoso, S. A., de Paula, S. O., Zanuncio, J. C., de Oliveira, L. L. Bacteriophage Isolated from Sewage Eliminates and Prevents the Establishment of Escherichia Coli Biofilm. Adv Pharm Bull. 2018, 8(1): 85-95.
Lee, Y. D., Park, J. H. Characterization and application of phages isolated from sewage for reduction of Escherichia coli O157:H7 in biofilm. LWT-Food Sci Technol. 2015.60(1): 571-577.
Ryan, E. M., Alkawareek, M. Y., Donnelly, R. F., Gilmore, B. F. Synergistic phage-antibiotic combinations for the control of Escherichia coli biofilms in vitro. FEMS Immunol Med Microbiol. 2012.65(2): 395-398.
Breyne, K., Honaker, R. W., Hobbs, Z., Richter, M., Żaczek, M., Spangler, T., Steenbrugge, J., Lu, R., Kinkhabwala, A., Marchon, B., Meyer, E., Mokres, L. Efficacy and Safety of a Bovine-Associated Staphylococcus aureus Phage Cocktail in a Murine Model of Mastitis. Front Microbiol. 2017.8:2348.
Geng, H., Zou, W., Zhang, M., Xu, L., Liu, F., Li, X., Wang, L., Xu, Y. Evaluation of phage therapy in the treatment of Staphylococcus aureus-induced mastitis in mice. Folia Microbiol (Praha). 2020.65(2): 339-351.
de Silva Duarte, V., Dias, R. S., Kropinski, A. M., Campanaro, S., Treu, L., Siqueira, C., Vieira, M. S., da Silva Paes, I., Santana, G. R., Martins, F., Crispim, J. S., da Silva Xavier, A., Ferro, C. G., Vidigal, P. M. P., da Silva, C. C., de Paula, S. O. Genomic analysis and immune response in a murine mastitis model of vB_EcoM-UFV13, a potential biocontrol agent for use in dairy cows. Sci Rep. 2018, 8(1): 6845.
Iwano, H., Inoue, Y., Takasago, T., Kobayashi, H., Furusawa, T., Taniguchi, K., Fujiki, J., Yokota, H., Usui, M., Tanji, Y., Hagiwara, K., Higuchi, H., Tamura, Y. Bacteriophage OSA012 Has a Broad Host Range against Staphylococcus aureus and Effective Lytic Capacity in a Mouse Mastitis Model. Biology (Basel). 2018, 7(1): pii: E8.
O'Flaherty, S., Ross, R. P., Flynn, J., Meaney, W. J., Fitzgerald, G. F., and Coffey, A. Isolation and characterisation of two anti-staphylococcal bacteriophages specific for pathogenic Staphylococcus aureus associated with bovine infections. Lett Appl Microbiol. 2005,41,482-486.
U.S. Pat. No. 8,043,613B2
Patent No. W02017087909A1
Patent No. KR20180042748A
Patent No. KR102003770B1
Mainau, E., Temple, D., Manteca, X. Welfare issues related to mastitis in dairy cows. Farm Animal Welfare Education Centre McHugh D J. 2014. A Guide to Seaweed Industry; p. 61-65.
Su, M. T., Tyamagondlui, V. V., Bodmer, R. Large- and small-scale preparation of bacteriophage lambda lysate and DNA. BioTechniques. 1998.25: 44-46.
Werthen, M., Henriksson, L., Jensen, P. Ø., Sternberg, C., Givskov, M., Bjamsholt, T. An in vitro model of bacterial infections in wounds and other soft tissues. APMIS. 2010.118: 156-64.

1. A preparation for use in prevention or treatment of bacterial infections in dairy cattle, in particular infections caused by E. coli and/or S. aureus, intended for intramammary administration during lactation or dry period, whereby said preparation has a form of gel obtained from an aqueous solution containing iota-carrageenan concentration ranging from 0.2 to 4.0% w/v and bacteriophages with at least 10⁷ PFU/g count.

2. A bacteriophage preparation for use according to claim 1, characterised in that it contains bacteriophages specific to E. coli and S. aureus bacteria strains causing infections in dairy cattle.

3. A bacteriophage preparation for use according to claim 1, characterised in that it contains bacteriophages with at least 10⁸ PFU/g count.

4. A bacteriophage preparation for use according to claim 1, characterised in that it contains a bacteriophage strain selected among those deposited in the Polish Collection of Microorganisms at the following deposit numbers: F/00152 (303Ecol101PP strain), F/00153 (308Ecol101PP strain), F/00154 (310Ecol104PP strain), F/00155 (348Ecol098PP strain), F/00151 (241Ecol014PP strain), F/00148 (351Saur083PP strain), F/00149 (355Saur083PP strain), F/00150 (357Saur119PP strain) or any mixture of these microorganisms.

5. A bacteriophage preparation for use according to claim 1, characterised in that it is intended for treating or preventing mastitis in dairy cattle.

6. The method of obtaining a bacteriophage preparation for intramammary administration to breeding animals, characterised in that:
an aqueous solution of iota-carrageenan is obtained at 0.4-8.0% w/v concentration,
an aqueous solution is obtained, containing bacteriophages with at least 10⁷ PFU/mL count, preferably at least 10⁸ PFU/mL and most preferably at least 4×10⁸ PFU/mL,
both solutions are mixed, preferably in sterile conditions, at temperatures ranging from 33° C. to 40° C., preferably 36° C., and then the solution is left for gelling.

7. Method according to claim 6, characterised in that the bacteriophage preparation described in claims 1-5 is the obtained preparation.

8. A bacteriophage strain selected among those deposited in the Polish Collection of Microorganisms (PCM) at the following deposit numbers: F/00152 (303Ecol101PP strain), F/00153 (308Ecol101PP strain), F/00154 (310Ecol104PP strain), F/00155 (348Ecol098PP strain), F/00151 (241Ecol014PP strain), F/00148 (351Saur083PP strain), F/00149 (355Saur083PP strain), F/00150 (357Saur119PP strain) or any mixture of these microorganisms.

9. A bacteriophage strain according to claim 8 for use in prevention or treatment of bacterial infections in dairy cattle, in particular infections caused by E. coli and/or S. aureus.

10. A bacteriophage for use according to claim 9, characterised in that it is intended for treating or preventing mastitis in dairy cattle.

11. A use an iota-carrageenan fraction as a gel delivery carrier to produce bacteriophage preparations intended for intramammary administration, whereby preferably the carrageenan is used in the form of aqueous solution at 0.4-8.0% w/v concentration.
